(19) United States
(12) Patent Application Publication
(10) Pub. No.: US 2011/0002862 A1
(43) Pub. Date: Jan. 6, 2011

(54) Method For The Production Of Polymers By Means Of Ion Exchange

(75) Inventors: Roman Morschhaeuser, Mainz (DE); Thomas Lindner, Mannheim (DE); Barbara Dueker, Mainz (DE); Ute Back, Blankenbach (DE); Matthias Loeffler, Idstein (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT FINANCE (BVI) LIMITED, Tortola (VG)

(21) Appl. No.: 12/866,289

(22) PCT Filed: Feb. 5, 2009
(86) PCT No.: PCT/EP09/00767

§ 371 Date: Aug. 5, 2010

(30) Foreign Application Priority Data
Feb. 8, 2008 (DE) 102008008179.5

(51) Int. Cl. A61K 8/81; B01J 47/00; A61Q 1/02

(52) U.S. Cl. 424/63; 521/38

(57) ABSTRACT

The invention relates to a method for the production of polymers, comprising one or more of the recurring structural units of formula (1),

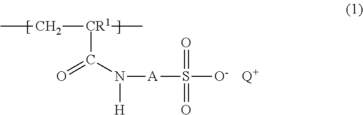

where R¹ is hydrogen, methyl, or ethyl, A is C₁-C₈-alkylene, Q⁺ is H⁺, NH₄⁺, Na⁺, K⁺, ½ Mg⁺⁺, ½ Ca⁺⁺, or ½ Zn⁺⁺, and where Q⁺ has a different meaning than H⁺ or NH₄⁺ in 50 to 100 mol % of the structural units of formula (1), and one or more structural units derived from one or more further monomers, characterized in that the corresponding salts of said polymers are suspended in an organic suspension, where however the cations Q⁺ in the structural units of the formula (1) are NH₄⁺ and optionally also H⁺, and an ion exchange is carried out by adding one or more bases comprising Na⁺, K⁺, Mg⁺⁺, Ca⁺⁺, or Zn⁺⁺ ions.

The present invention relates to a method of producing water-soluble or water-swellable alkali metal, alkaline earth metal or zinc salts of polymers based on acrylamidoalkylsulfonic acids or derivatives thereof, for example alkali metal, alkaline earth metal or zinc salts of crosslinked homopolymers of acrylamidoalkylsulfonic acids or of copolymers containing, in addition to structural units derived from acrylamidoalkylsulfonic acids, additional structural units derived from cyclic and/or linear N-vinylcarboxamides or derived from esters of (meth)acrylic acid with fatty alcohol polyglycol ethers. The alkali metal, alkaline earth metal or zinc salts of these polymers are obtained by ion exchange from the more readily accessible ammonium salts with bases of the corresponding metals. The invention further relates to the polymers obtainable by the method of the present invention, to the use of these polymers as a thickener or stabilizer, and also to self-tanning agents containing one or more of these polymers and dihydroxyacetone.

Water- or solvent-containing multicomponent systems such as solutions, emulsions or suspensions are frequently adjusted to higher viscosities, or thickened, for reasons of cost or performance or for stability reasons.

For instance, increasing the viscosity of the external or internal phase of emulsions or suspensions is a way to distinctly prolong the time before the components of such a system separate, which manifests itself in a longer shelf life. Increasing the viscosity also improves for many products their ability to be distributed uniformly over uneven surfaces in particular. This applies particularly to skincare agents and pharmaceutical ointments on the skin. There are many industrial products such as wallpaper strippers, paint strippers or aircraft deicers where the increased viscosity prevents premature run-off from the surface to be treated. The greater uniformity of distribution and the extended contact time thus serve to increase efficacy. In addition to the performance advantages mentioned, the high viscosity of such preparations also offers further advantages in manufacture, packaging, bottling and storage and also in transportation.

In general, the rheological properties in the manufacture and/or formulation of cosmetic, pharmaceutical or industrial preparations are a decisive criterion for the use of these products in commercial practice. The thickeners used should provide adequate thickening even when used at very low use levels.

To adjust the rheological properties of aqueous or solvent-containing systems, emulsions or suspensions, a multiplicity of different systems are discussed in the technical literature. Well-known examples are cellulose ethers and other cellulose derivatives (e.g., carboxymethylcellulose, hydroxyethylcellulose), gelatin, starch and starch derivatives, sodium alginates, fatty acid polyethylene glycol esters, agar agar, tragacanth or dextrins. Various synthetic polymers are used, examples being polyvinyl alcohols, polyacrylamides, polyacrylic acid and various salts of polyacrylic acid, polyvinylpyrrolidone, polyvinyl methyl ether, polyethylene oxides, copolymers of maleic anhydride and vinyl methyl ether, and also diverse mixtures and copolymers of the compounds recited above.

However, the compounds mentioned do have a variety of disadvantages in use. For instance, cellulose derivatives or, more generally, materials based on natural resources, and the formulations resulting therefrom, are very vulnerable to bacteria. In use, they mostly become apparent through the formation of unpleasant “stringy” gels. Fatty acid polyethylene glycol esters tend to hydrolyze in the presence of water and the resulting insoluble fatty acids give rise to unwanted cloudiness. Thickeners of natural origin (e.g., agar agar or tragacanth) vary greatly in composition, depending on their origin.

EP 1 116 733 describes high thickening ability on the part of copolymers obtained by polymerization of monomers selected from acrylamidopropyl-methylenesulfonic acid, salts of acrylamidopropylmethylenesulfonic acid, acrylamide, N-vinylformamide, N-vinylmethylacetamide and sodium methallylsulfonate.

EP 1 069 142 discloses copolymers obtained by free-radical copolymerization of one or more macromonomers selected from the group consisting of esters of (meth)acrylic acid with alkyl ethoxylates comprising from 5 to 80 ethylene oxide units and (C₁₀-C₂₂)-alkyl radicals, and one or more olefinically unsaturated comonomers selected from the group consisting of acrylamidopropylmethylenesulfonic acid, sodium and ammonium salts of acrylamidopropylmethylenesulfonic acid, acrylamide, N-vinylformamide, N-vinylmethylacetamide and sodium methallylsulfonate.

Representatives of the copolymers mentioned in EP 1 116 733 and EP 1 069 142 are commercially available as ammonium salts under the trade names of Aristoflex® AVC and Aristoflex® HMB (Clariant). Similarly, the ammonium salt of crosslinked acrylamidopropylmethylenesulfonic acid is also commercially available.

The ammonium salts of these polymers have the disadvantage that they are unstable in alkaline compositions having a pH>8.5, releasing ammonia.

The abovementioned commercially available polymers are prepared, as described in EP 1 116 733 and EP 1 069 142, in a precipitation polymerization in tert-butanol at a water content of less than 10% by weight. This process cannot use an alkali or alkaline earth metal of acrylamidopropylmethylenesulfonic acid as a monomer since these are only available as an approximately 50% by weight aqueous solution. The water in the 50% by weight aqueous acrylamidopropylmethylenesulfonate salt solution would lead to gelling in the existing process, since the resulting polymers have a substantial thickening effect on aqueous systems. The 100% sodium salt of the monomer is not stable, however.

Accordingly, alkali metal, alkaline earth metal or zinc salts of the polymers disclosed in the patent applications mentioned above are not readily accessible by this method.

It is an object of the present invention to provide a method whereby alkali metal, alkaline earth metal or zinc salts of polymers based on acrylamidoalkylsulfonic acids or derivatives thereof are simple to produce on a large industrial scale.

We have found that this object is achieved by a method which is a simple way of obtaining alkali metal, alkaline earth metal or zinc salts, preferably the Na⁺, K⁺, Mg⁺⁺, Ca⁺⁺ or Zn⁺⁺ salts, of these polymers and which proceeds from the corresponding ammonium salts of the polymers, suspends them in an organic solvent and subjects them to an ion exchange by adding one or more bases containing Na⁺, K⁺, Mg⁺⁺, Ca⁺⁺ or Zn⁺⁺ ions.

The invention accordingly provides a method of producing polymers containing
a) one or more of the structural repeat units of formula (1)

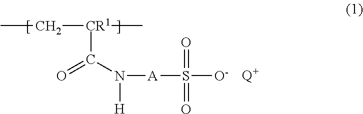

where R¹ is hydrogen, methyl or ethyl, A is C₁-C₈-alkylene, Q⁺ is H⁺, NH₄⁺, Na⁺, K⁺, ½Mg⁺⁺, ½ Ca⁺⁺ or ½Zn⁺⁺ subject to the proviso that in from 50 to 100 mol %, preferably in from 80 to 100 mol %, more preferably in from 90 to 100 mol %, even more preferably in from 95 to 100 mol % and yet even more preferably in from 98 to 100 mol % of the structural units of formula (1) Q⁺ has a meaning other than H⁺ or NH₄⁺,
b) optionally one or more of the structural repeat units of formula (2)

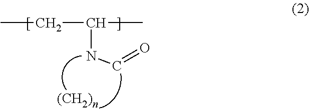

where n is an integer from 2 to 9,
c) optionally one or more of the structural repeat units of formula (3)

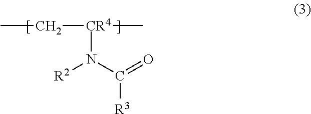

where R², R³ and R⁴ may be the same or different and are each hydrogen, a linear or branched alkyl group having from 1 to 30, preferably from 1 to 20 and more preferably from 1 to 12 carbon atoms or a linear or branched mono- or polyunsaturated alkenyl group having from 2 to 30, preferably from 2 to 20 and more preferably from 2 to 12 carbon atoms,
d) optionally one or more of the structural repeat units derived from monomers of formula (4)

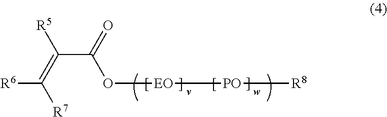

where R⁵ is H or —CH₃, R⁶ and R⁷ are each independently H or —CH₃, preferably H, R⁸ is a linear or branched alkyl group having from 1 to 30, preferably from 8 to 22 and more preferably from 12 to 18 carbon atoms, a linear or branched mono- or polyunsaturated alkenyl group having from 2 to 30, preferably from 8 to 22 and more preferably from 12 to 18 carbon atoms, or a cycloaliphatic, arylaliphatic or aromatic hydrocarbyl radical having from 6 to 30 carbon atoms, EO is an ethylene oxide unit, PO is a propylene oxide unit, v and w are each independently from 0 to 500, the sum total of v and w is on average ≧1, and the distribution of the EO and PO units across the -(-[EO]ᵥ-[PO]_{w}-)- group can be random, blocklike, alternating or gradientlike, and
e) optionally one or more crosslinking structural units derived from monomers having two or more olefinic double bonds,
   with the proviso that the polymers contain one or more structural units selected from b) to e),
   which method comprises suspending the corresponding salts of these polymers, in which the Q⁺ cations in the structural units of formula (1) have the meanings of NH₄⁺ and additionally, where appropriate, H⁺, as a starting material in an organic solvent and adding one or more bases containing Na⁺, K⁺, Mg⁺⁺, Ca⁺⁺ or Zn⁺⁺ ions to effect an ion exchange.

The distribution of the structural units within the polymers produced or obtainable by the method of the present invention may be random, blocklike, alternating or gradientlike.

The molecular weight of the polymers produced or obtainable by the method of the present invention is preferably in the range from 10³ to 10⁹ g/mol, more preferably in the range from 10⁴ to 10⁷ g/mol and even more preferably in the range from 5×10⁴ to 5×10⁶ g/mol.

The polymers produced or obtainable by the method of the present invention are water-soluble or water-swellable.

The method of the present invention proceeds from polymers containing one or more structural units of formula (1) with NH₄⁺ and, if appropriate, H⁺ as counterion and one or more structural units selected from b) to e) as starting materials, the degree of neutralization of the acid from which the structural unit of formula (1) is derived preferably being in the range from 50 to 100%, more preferably in the range from 80 to 100%, even more preferably in the range from 90 to 100%, yet even more preferably in the range from 95 to 100% and yet still even more preferably in the range from 98 to 100%.

More particularly, the starting material contains preferably from 50 to 100 mol % of the structural units of formula (1) with NH₄⁺ as counterion and from 0 to 50 mol % of the structural units of formula (1) with H⁺ as counterion, more preferably from 80 to 100 mol % of the structural units of formula (1) with NH₄⁺ as counterion and from 0 to 20 mol % of the structural units of formula (1) with H⁺ as counterion, even more preferably from 90 to 100 mol % of the structural units of formula (1) with NH₄⁺ as counterion and from 0 to 10 mol % of the structural units of formula (1) with H⁺ as counterion, yet even more preferably from 95 to 100 mol % of the structural units of formula (1) with NH₄⁺ as counterion and from 0 to 5 mol % of the structural units of formula (1) with H⁺ as counterion, and yet still even more preferably from 98 to 100 mol % of the structural units of formula (1) with NH₄ as counterion and from 0 to 2 mol % of the structural units of formula (1) with H⁺ as counterion.

An extremely preferable embodiment of the invention is a method wherein the starting material contains 100 mol % of the structural units of formula (1) with NH₄⁺ as counterion.

The organic solvent used in the method of the present invention is preferably a protic solvent, more preferably tert-butanol.

The bases used in the method of the present invention are preferably selected from hydroxides and alkoxylates and more preferably from hydroxides and alkoxylates of Na⁺, K⁺ and/or Zn⁺⁺. The base NaOH is even more preferable.

The method of the present invention is preferably carried out at a temperature in the range from 25 to 100° C., more preferably in the range from 40 to 100° C. and even more preferably in the range from 60 to 100° C. When the solvent used in the method of the present invention has a boiling point of less than 100° C., as in the case of tert-butanol for example, the method is preferably carried out at a temperature in the range from 25° C. to the boiling point of the solvent used, more preferably in the range from 40° C. to the boiling point of the solvent used, even more preferably in the range from 60° C. to the boiling point of the solvent used and yet even more preferably at the boiling point of the solvent used.

The method of the present invention is preferably carried out at atmospheric pressure.

The method of the present invention is preferably carried out by passing an inert gas, preferably nitrogen and more preferably at a flow rate in the range from 40 to 80 l/h of inert gas, through.

The method of the present invention is preferably carried out at a pH in the range from 7.5 to 8.5.

The method of the present invention is preferably carried out by suspending the starting material in an organic protic solvent, preferably in tert-butanol, preferably by vigorous stirring, preferably at 40 to 60° C., preferably passing an inert gas and more preferably nitrogen through and adding one or more bases containing Na⁺, K⁺, Mg⁺⁺, Ca⁺⁺ or Zn⁺⁺ ions, preferably NaOH, more preferably concentrated NaOH (50% by weight in water), by metered addition. As the pH rises to 7.5-8.5, ammonia is formed, this ammonia being preferably continuously removed by the inert gas stream until ammonia egress is no longer measurable and the ion exchange of ammonium ions for Na⁺, K⁺, Mg⁺⁺, Ca⁺⁺ or Zn⁺⁺ ions is quantitative.

The method of the present invention is more preferably carried out by suspending the starting material in an organic protic solvent, preferably in tert-butanol, preferably by stirring, adding one or more bases selected from hydroxides and alkoxylates of Na⁺, K⁺, Mg⁺⁺, Ca⁺⁺ or Zn⁺⁺, preferably one or more bases selected from hydroxides and alkoxylates of Na⁺, K⁺ or Zn⁺⁺, more preferably NaOH, by metered addition, and performing the method at a temperature in the range from 25 to 100° C. and when a solvent having a boiling point of less than 100° C. is used at a temperature in the range from 25° C. to the boiling point of the solvent used, preferably at a temperature in the range from 40 to 100° C. and when a solvent having a boiling point of less than 100° C. is used at a temperature in the range from 40° C. to the boiling point of the solvent used, and preferably by passing an inert gas, more preferably nitrogen and even more preferably at a flow rate in the range from 40 to 80 l/h of inert gas, through, at a pH in the range from 7.5 to 8.5.

The method of the present invention is even more preferably carried out by
a) the corresponding predominantly solid ammonium salts of the polymers, i.e., the starting material, being suspended in a protic solvent by stirring and preferably heated up to 40-60° C.,
b) preferably passing an inert gas, more preferably nitrogen and even more preferably at a flow rate of 60 l/h, through via an inlet tube,
c) metering bases containing Na⁺, K⁺, Mg⁺⁺, Ca⁺⁺ or Zn⁺⁺ ions into the reactor, preferably at a dropping rate of from 3 to 9 g/minute,
d) raising the internal temperature in the reactor to boiling heat,
e) maintaining the pH at 7.5 to 8.5 via the metering rate,
f) preferably trapping resulting ammonia with an acid, more preferably with sulfuric acid and even more preferably with 25% by weight of sulfuric acid in water,
g) preferably stirring under reflux on completion of the metered addition, more preferably stirring under reflux for 1.5 hours,
h) cooling the suspension down, and
i) drying the batch, preferably in a vacuum cabinet at from 30 to 50° C. and from 10 to 100 and preferably from 80 to 100 mbar.

The method of the present invention is yet even more preferably carried out by
j) the corresponding predominantly solid ammonium salts of the polymers, i.e., the starting material, being suspended in a protic solvent, preferably in tert-butanol, by stirring and preferably heated up to 40-60° C.,
k) passing an inert gas, preferably nitrogen and more preferably at a flow rate of 60 l/h, through via an inlet tube,
l) metering bases containing Na⁺, K⁺, Mg⁺⁺, Ca⁺⁺ or Zn⁺⁺ into the reactor, preferably at a dropping rate of from 3 to 9 g/minute,
m) raising the internal temperature in the reactor to boiling heat,
n) maintaining the pH at 7.5 to 8.5 via the metering rate,
o) preferably trapping resulting ammonia with an acid, more preferably with sulfuric acid and even more preferably with 25% by weight of sulfuric acid in water,
p) preferably stirring under reflux on completion of the metered addition, more preferably stirring under reflux for 1.5 hours,
q) cooling the suspension down, and
r) drying the batch, preferably in a vacuum cabinet at from 30 to 50° C. and from 10 to 100 and preferably from 80 to 100 mbar.

The method of the present invention more preferably employs bases of the alkali and alkaline earth metals selected from NaOH, KOH, sodium alkoxylates, potassium alkoxylates, preferably sodium tert-butoxide or potassium tert-butoxide, and also zinc compounds selected from Zn(OH)₂ and zinc alkoxylates.

A particularly preferred embodiment of the invention is a method of producing sodium salts of the polymers.

When bases with monovalent cations are used for the method of the present invention, the molar ratio of cations Q⁺ to be exchanged in the polymer, or starting material, to monovalent cations of the base used for the ion exchange is preferably in the range from 1.0:0.5 to 2.0, more preferably in the range from 1.0:0.80 to 1.0, even more preferably in the range from 1.0:0.90 to 1.0, yet even more preferably in the range from 1.0:0.95 to 1.0 and yet still even more preferably in the range from 1.0:0.98 to 1.0. When bases with divalent cations are used for the method of the present invention, the corresponding molar ratio is preferably in the range from 1.0:0.25 to 1.0, more preferably in the range from 1.0:0.4 to 0.5, even more preferably in the range from 1.0:0.45 to 0.5, yet even more preferably in the range from 1.0:0.475 to 0.5 and yet still even more preferably in the range from 1.0:0.49 to 0.5.

The amount of polymer starting material in the solvent in the method of the present invention is preferably in the range from 5.0% to 80.0% by weight, more preferably in the range from 7.0% to 50.0% by weight and even more preferably in the range from 10.0% to 30.0% by weight.

Preference is further given to methods of the present invention wherein the structural units of formula (1) are derived from 2-acrylamido-2-methylpropanesulfonic acid.

Preference is further given to methods of the present invention wherein the structural units of formula (2) are derived from N-vinylpyrrolidone.

Preference is further given to methods of the present invention wherein R², R³ and R⁴ in the structural units of formula (3) are each independently hydrogen or methyl.

Preference is further given to methods of the present invention wherein R³ in the structural units derived from monomers of formula (4) is a linear or branched alkyl group having from 8 to 22 and preferably from 12 to 18 carbon atoms, or is a linear or branched mono- or polyunsaturated alkenyl group having from 8 to 22 and preferably from 12 to 18 carbon atoms. Among these, preference is given in turn to those methods of the present invention wherein R⁸ in the structural units derived from the monomers of formula (4) is an alkyl group.

In a further preferred embodiment of the method of the present invention, the sum total of v and w in the structural units derived from the monomers of formula (4) is on average in the range from 1 to 40, preferably in the range from 3 to 40, more preferably in the range from 5 to 30, even more preferably in the range from 8 to 25 and yet even more preferably in the range from 10 to 25. In these structural units derived from the monomers of formula (4), v and w are each independently preferably from 0 to 40, more preferably from 0 to 30 and even more preferably from 0 to 25. Among these, preference is given in turn to those methods of the present invention wherein w in the structural units derived from the monomers of formula (4) is 0.

In a particularly preferred embodiment of the method of the present invention, the structural units derived from the monomers of formula (4) are derived from

C₁₀-C₁₈ fatty alcohol polyglycol ethers with 8 EO units (Genapol® C-080)
C₁₁ oxo process alcohol polyglycol ethers with 8 EO units (Genapol® UD-080)
C₁₂-C₁₄ fatty alcohol polyglycol ethers with 7 EO units (Genapol® LA-070)
C₁₂-C₁₄ fatty alcohol polyglycol ethers with 11 EO units (Genapol® LA-110)
C₁₆-C₁₈ fatty alcohol polyglycol ethers with 8 EO units (Genapol® T-080)
C₁₆-C₁₈ fatty alcohol polyglycol ethers with 11 EO units (Genapol® T-110)
C₁₆-C₁₈ fatty alcohol polyglycol ethers with 15 EO units (Genapol® T-150)
C₁₆-C₁₈ fatty alcohol polyglycol ethers with 20 EO units (Genapol® T-200)
C₁₆-C₁₈ fatty alcohol polyglycol ethers with 25 EO units (Genapol® T-250)
C₁₈-C₂₂ fatty alcohol polyglycol ethers with 25 EO units and/or
iso C₁₆-C₁₈ fatty alcohol polyglycol ethers with 25 EO units,
wherein these recited structures represent preferred structures for the substructure —O—(-[EO]ᵥ-[PO]_{w}-)—R⁸ of formula (4) and the alcohols HO—(-[EO]ᵥ-[PO]_{w}-)—R⁸ underlying these substructures are more preferably esterified with acrylic acid or methacrylic acid. The Genapol® grades are products of Clariant.

In a further preferred embodiment of the method of the present invention, v in the structural units derived from the monomers of formula (4) is from 1 to 30, preferably from 3 to 25, and w is from 1 to 30 and preferably from 3 to 15.

The one or more crosslinking structural units of component e) differ from the structural units of components a) to d).

Preference is further given to methods of the present invention wherein the crosslinking structural units of component e) are derived from allyl acrylate or methacrylate, diallyl maleate, dipropylene glycol diallyl ether, polyglycol diallyl ether, triethylene glycol divinyl ether, hydroquinone diallyl ether, tetraallyloxyethane or other allyl or vinyl ethers of multifunctional alcohols, tetraethylene glycol diacrylate, polyethylene glycol dimethacrylate having on average 9 ethylene oxide units in the polyethylene glycol unit, triallylamine, triallyl cyanurate, trimethylolpropane diallyl ether, methylenebisacrylamide, methylenebismethacrylamide, trimethylolpropane triacrylate (TMPTA), trimethylolpropane trimethacrylate (TMPTMA) or divinylbenzene.

It is particularly preferable for the crosslinking structural units of component e) to be derived from monomers of formula (5),

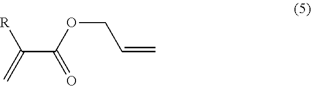

where R is hydrogen, methyl or ethyl, from trimethylolpropane triacrylate, from trimethylolpropane trimethacrylate or from polyethylene glycol dimethacrylate having on average 9 ethylene oxide units in the polyethylene glycol unit. It is even more preferable for the crosslinking structural units of component e) to be derived from trimethylolpropane triacrylate, from trimethylolpropane trimethacrylate or from polyethylene glycol dimethacrylate having on average 9 ethylene oxide units in the polyethylene glycol unit.

Preferably, the polymers produced or obtainable by the method of the present invention consist of structural units selected from the structural units of components a) to e).

In a preferred embodiment of the invention, the polymers produced or obtainable by the method of the present invention do not contain any crosslinking structural units of component e).

In a further preferred embodiment of the invention, the polymers produced or obtainable by the method of the present invention contain up to 8%, preferably from 0.01% to 8%, more preferably from 0.01% to 5% and even more preferably from 0.02% to 3% by weight of one or more crosslinking structural units of component e).

A particularly preferred embodiment of the invention is a method of producing polymers containing, preferably consisting of,
a) from 49.99% to 98.99% by weight of one or more of the structural repeat units of formula (1),
b) from 1% to 50% by weight of one or more of the structural repeat units of formula (2), and
e) from 0% to 8% and preferably from 0.01% to 5% by weight of one or more crosslinking structural units derived from monomers having two or more olefinic double bonds.

Of these, preference is in turn given to methods of the present invention for producing polymers containing, preferably consisting of,
a) from 69.5% to 97.5% and preferably from 84.5% to 96.5% by weight of one or more of the structural repeat units of formula (1), preferably derived from 2-acrylamido-2-methylpropanesulfonic acid,
b) from 2% to 30% and preferably from 3% to 15% by weight of one or more of the structural repeat units of formula (2), preferably derived from N-vinylpyrrolidone, and
e) from 0.2% to 3% and preferably from 0.5% to 2% by weight of one or more crosslinking structural units derived from monomers having two or more olefinic double bonds.

A further particularly preferred embodiment of the invention is a method of producing polymers containing, preferably consisting of,
a) from 49.99% to 98.99% by weight of one or more of the structural repeat units of formula (1),
d) from 1% to 50% by weight of one or more of the structural repeat units derived from monomers of formula (4), and
e) from 0% to 8% and preferably from 0.01% to 5% by weight of one or more crosslinking structural units derived from monomers having two or more olefinic double bonds.

Within this particularly preferred embodiment, preference is in turn given to a method of producing polymers containing, preferably consisting of,
a) from 69.5% to 97.5% and preferably from 80% to 96.5% by weight of one or more of the structural repeat units of formula (1), preferably derived from 2-acrylamido-2-methylpropanesulfonic acid, and
d) from 2.5% to 30.5%, preferably from 3.5% to 20% by weight of one or more of the structural repeat units derived from monomers of formula (4) where R⁵ is H or —CH₃, R⁶ and R⁷ are each independently H or —CH₃, preferably H, R⁸ is a linear or branched alkyl group having from 8 to 22 and preferably from 12 to 22 carbon atoms, or is a linear or branched mono- or polyunsaturated alkenyl group having from 8 to 22 and preferably from 12 to 22 carbon atoms, v is from 1 to 30 and preferably from 3 to 25, and w is 0.

Within this particularly preferred embodiment of the invention, preference is in turn given to a method of producing polymers containing, preferably consisting of,
a) from 69.5% to 97.5% and preferably from 79.5% to 96.5% by weight of one or more of the structural repeat units of formula (1), preferably derived from 2-acrylamido-2-methylpropanesulfonic acid,
d) from 2% to 30%, preferably from 3% to 20% by weight of one or more of the structural repeat units derived from monomers of formula (4) where R⁵ is H or —CH₃, R⁶ and R⁷ are each independently H or —CH₃, preferably H, R⁸ is a linear or branched alkyl group having from 8 to 22 and preferably from 12 to 22 carbon atoms, or is a linear or branched mono- or polyunsaturated alkenyl group having from 8 to 22 and preferably from 12 to 22 carbon atoms, v is from 1 to 30 and preferably from 3 to 25, and w is 0, and
e) from 0.2% to 3% and preferably from 0.5% to 2% by weight of one or more crosslinking structural units derived from monomers having two or more olefinic double bonds.

A further particularly preferred embodiment of the invention is a method of producing polymers containing, preferably consisting of,
a) from 92% to 99.99% and preferably from 95% to 99.99% by weight of one or more of the structural repeat units of formula (1), and
e) from 0.01% to 8% and preferably from 0.01% to 5% by weight of one or more crosslinking structural units derived from monomers having two or more olefinic double bonds.

The invention further provides the polymers obtainable by the method of the present invention.

In a further preferred embodiment of the invention, the polymers produced or obtainable by the method of the present invention contain one or more structural units of formula (1) and one or more structural units selected from the structural units of formula (2), the structural units derived from the monomers of formula (4) and the crosslinking structural units derived from monomers having two or more olefinic double bonds, and in a particularly preferred embodiment of the invention the polymers produced or obtainable by the method of the present invention consist of the structural units just mentioned.

The polymers obtainable by the method of the present invention have a substantial thickening effect, more particularly in cosmetic or pharmaceutical compositions, preferably at polymer concentrations in the range from 0.1% to 5% by weight, more preferably from 0.5% to 2% by weight and even more preferably from 0.7 to 1% by weight, based on the final compositions. Viscosities of more than 60 000 mPa·s can be reached at room temperature in deionized water at a pH from 6 to 7.

The polymers obtainable by the method of the present invention, in addition to their thickening effect, also have a stabilizing effect, more particularly in cosmetic or pharmaceutical compositions.

The polymers obtainable by the method of the present invention display but relatively minor changes in viscosity over a wide pH range, preferably over a pH range from 2.5 to 12 and more preferably from 2.5 to 11. In addition, they retain their good water solubility in the formulations, more particularly in the cosmetic or pharmaceutical compositions, and are easily washed off the skin. Their thickening and stabilizing properties are also effective in aqueous, aqueous-alcoholic, alcoholic and/or glycol-containing solutions. They are UV stable and also stable over a wide temperature range.

Another advantage is the very good stability of the polymers obtainable by the method of the present invention in alkaline compositions, preferably at from pH 8 to pH 12, more preferably at from pH 9 to pH 12 and even more preferably at from pH 9 to pH 11.

Varying the monomers and also the fraction of crosslinker gives polymers useful as thickeners in both oil-in-water emulsions and water-in-oil emulsions at from pH 2.5 to pH 12, preferably from pH 2.5 to pH 11.

The present invention accordingly further provides for the use of the polymers obtainable by the method of the present invention as a thickener or stabilizer, preferably in cosmetic or pharmaceutical compositions. The use as a thickener is preferred.

In a preferred embodiment of the invention, the thickened and stabilized, preferably the thickened, compositions are present in the form of aqueous systems, emulsions or dispersions.

Depending on whether lotions having comparatively low viscosities or creams and ointments having high viscosities are to be produced, emulsions contain an oily substance consisting essentially of one or more emulsifiers and an oil phase in amounts ranging from 5% to 95% and preferably from 25% to 85% by weight, and water in the amount missing from 100% by weight.

Useful oily substances include vegetable, animal, mineral and synthetic oils, for example Guerbet alcohols having from 6 to 18 and preferably from 8 to 10 carbon atoms, esters of linear C₆-C₁₃ fatty acids with linear C₆-C₂₀ fatty alcohols, esters of branched C₆-C₁₃ carboxylic acids with linear C₆-C₂₀ fatty alcohols, esters of linear C₆-C₁₈ fatty acids with branched alcohols, more particularly 2-ethylhexanol, esters of linear and/or branched fatty acids with polyhydric alcohols (such as, for example, dimerdiol or trimerdiol) and/or Guerbet alcohols, triglycerides based on C₆-C₁₀ fatty acids, vegetable oils, branched primary alcohols, substituted cyclohexanes, Guerbet carbonates, dialkyl ethers and/or aliphatic or aromatic hydrocarbons.

The emulsions can be present as skincare agents, for example day creams, night creams, care creams, nourishing creams, body lotions, ointments and the like, and contain, as further auxiliary and added substances, coemulsifiers, superfatting agents, fats, waxes, stabilizers, biogenic actives, glycerol, preservatives, pearlizing agents, dyes and fragrances.

Useful superfatting agents include substances such as, for example, polyethoxylated lanolin derivatives, lecithin derivatives, polyol fatty acid esters, monoglycerides and fatty acid alkanolamides, the latter also serving as foam stabilizers. Typical examples of fats are glycerides; and useful waxes include inter alia beeswax, paraffin wax or microwaxes, alone or combined with hydrophilic waxes, for example cetyl stearyl alcohol.

Useful stabilizers include metal salts of fatty acids, for example magnesium stearate, aluminum stearate and/or zinc stearate.

Examples of biogenic actives are plant extracts and vitamin complexes.

Useful preservatives include for example phenoxyethanol, formaldehyde solution, parabens, pentanediol or sorbic acid.

Useful pearlizing agents include for example glycol distearic acid esters such as ethylene glycol distearate, but also fatty acid monoglycol esters.

Useful dyes include the substances suitable and approved for cosmetic purposes, as listed for example in the publication “Kosmetische Färbemittel” [Cosmetic Colorants] of the Dye Commission of the German Research Community, Verlag Chemie, Weinheim, 1984, pp. 81-106.

The total fraction of auxiliary and added substances can amount to from 1% to 10% and preferably from 2% to 5% by weight, based on the agents, preferably the cosmetic or pharmaceutical compositions.

The agents, preferably the cosmetic or pharmaceutical compositions, can be produced in a conventional manner, i.e., for example, by hot, hot/cold or PIT emulsification.

Preference is given to the use of the polymers obtainable by the method of the present invention as a thickener or stabilizer, preferably as a thickener, in compositions, preferably in cosmetic or pharmaceutical compositions, whose pH is adjusted to a value above 7, preferably in the range from 8 to 12, more preferably in the range from 9 to 12 and even more preferably in the range from 9 to 11.

Preferred compositions are hair-smoothing agents at from pH 8 to pH 10, preferably at from pH 9 to pH 9.5, hair-removing agents, liquid body cleansers, preferably body cleansers containing fatty acid salts, more particularly those of stearic, palmitic and oleic acid, in which case the pH of these body cleansers can be in the range from 9 to 11 and preferably from 9.5 to 10.5.

Preference is further given to the use of the polymers obtainable by the method of the present invention as a thickener or stabilizer, preferably as a thickener, in alkaline lotions or creams for smoothing the skin or in alkaline hair colorants, more particularly in alkaline developer or coupler solutions.

The polymers obtainable by the method of the present invention are useful for thickening not just alkaline compositions but also compositions, preferably cosmetic or pharmaceutical compositions, having a pH of below 7, preferably in the range from 3 to 6.5, more preferably in the range from 3.5 to 5.5 and even more preferably in the range from 4 to 5. Discoloration of the compositions, as observed when ammonium salts of these polymers are used, does not occur in the presence of the polymers obtainable by the method of the present invention.

Preference is further given to the use of the polymers obtainable by the method of the present invention as a thickener or stabilizer, preferably as a thickener, in self-tanning agents containing from 0.1% to 10% by weight of dihydroxyacetone, based on the final agents, wherein these self-tanning agents are preferably adjusted to a pH in the range from 3.5 to 5.5 and more preferably in the range from 4 to 5.

The invention further provides self-tanning agents containing one or more polymers obtainable by the method of the present invention and from 0.1% to 10% by weight of dihydroxyacetone, based on the final agents, wherein these self-tanning agents are preferably adjusted to a pH in the range from 3.5 to 5.5 and more preferably in the range from 4 to 5. The amount in which the one or more polymers obtainable by the method of the present invention is or are present in these self-tanning agents is preferably in the range from 0.1% to 5% by weight and more preferably in the range from 0.5% to 2% by weight, based on the final agents.

The examples which follow are intended to more particularly elucidate possible uses for the polymers obtainable by the method of the present invention without limiting them thereto. The percentages are in all cases weight percentages (% by weight).

### EXAMPLE 1

### Aristoflex® AVC, Ion Exchange of NH₄⁺ for Na⁺

4432 g of Aristoflex® AVC (Ammonium AcryloyldimethyltaurateNP Copolymer, crosslinked, Clariant, VP: vinylpyrrolidone) are initially charged to a reactor fitted with impeller stirrer, pH electrode, temperature sensor, reflux condenser, inlet tube for N₂ and metering means for aqueous sodium hydroxide solution and while stirring at 330 revolutions/minute suspended in 16 450 g of tert-butanol and heated (N₂ stream 60 l/h). Starting at an internal temperature of 50° C., a peristaltic pump is used to meter 1329 g of NaOH (50% by weight in water, i.e., 664.5 g of NaOH in 664.5 g of water) into the reactor at a dropping rate of 4.9 g/minute. In the process, the internal temperature is raised to the boiling temperature.

The pH rises from an initial 4.5 and is maintained at between 7.5 and 8.5 via the metering rate.

The resulting ammonia is trapped via a wash bottle filled with sulfuric acid (25% by weight in water), as ammonium sulfate.

On completion of the metered addition the batch is stirred under reflux for 1.5 hours. For 30 minutes of that period, ammonia gas is driven out of the batch. The suspension is cooled down to 40° C. with stirring, and the batch is poured onto metal sheets and dried in a vacuum cabinet at 50° C. and 20 mbar for 2 days.

In the product obtained, 95 mol % of the ions to be exchanged in Aristoflex® AVC have been exchanged for Na⁺ ions.

### EXAMPLE 1A

Example 1 was repeated using 1371 g of NaOH (50% by weight in water, i.e., 685.5 g of NaOH in 685.5 g of water) instead of 1329 g. In the product obtained, ≧98 mol % of the ions to be exchanged in Aristoflex® AVC have been exchanged for Na⁺ ions.

### EXAMPLE 2

### Aristoflex® HMB, Ion Exchange of NH₄⁺ for Na⁺

100.0 g of Aristoflex® HMB (Ammonium Acryloyldimethyltaurate/Beheneth-25 Methacrylate, crosslinked, Clariant) are initially charged to a reactor fitted with anchor stirrer, pH electrode, temperature sensor, reflux condenser, inlet tube for N₂ and metering means for aqueous sodium hydroxide solution and while stirring at 330 revolutions/minute suspended in 400 g of tert-butanol and heated (N₂ stream: 60 l/h). Starting at an internal temperature of 50° C., a peristaltic pump is used to meter 30.3 g of NaOH (50% by weight in water, i.e., 15.15 g of NaOH in 15.15 g of water) into the reactor at a dropping rate of 0.50 g/minute. In the process, the internal temperature is raised to the boiling temperature.

The pH rises from an initial 3.3 and is maintained at between 7.5 and 8.5 via the metering rate.

The resulting ammonia is trapped via a wash bottle filled with sulfuric acid (25% by weight in water), as ammonium sulfate.

On completion of the metered addition the batch is stirred under reflux for 1.5 hours. For 15 minutes of that period, ammonia gas is driven out of the batch. The suspension is cooled down to 30° C. with stirring, and the batch is poured onto metal sheets and dried in a vacuum cabinet at 50° C. and 20 mbar overnight.

In the product obtained, ≧95 mol % of the ions to be exchanged in Aristoflex® HMB have been exchanged for Na⁺ ions.

The term “Beheneth-25 Methacrylate” in Aristoflex® HMB is to be understood as meaning that this copolymer contains structural units of formula (4) where R⁶ and R⁷ are H, R⁵ is CH₃, v is 25, w is 0 and R⁸ is behenyl.

### FORMULATION EXAMPLES

### Formulation Example 1

### O/W Self-Tanning Cream

| | | | |
|---|---|---|---|
| A | Hostaphat ® CC 100 | (Clariant) | 1.0% |
| | Cetyl Phosphate | | |
| | Glyceryl Stearate | | 0.5% |
| | Cetearyl Alcohol | | 0.5% |
| | Isohexadecane | | 8.0% |
| | Isopropyl Palmitate | | 7.0% |
| | SilCare ® Silicone 41M15 | (Clariant) | 1.0% |
| | Caprylyl Methicone | | |
| B | Sodium Acryloyldimethyltaurate/VP | | 1.5% |
| | Copolymer (crosslinked) | | |
| C | Water | | ad 100% |
| | Hostapon ® CGN | (Clariant) | 0.5% |
| | Sodium Cocoyl Glutamate | | |
| | Glycerol | | 5.0% |
| | Sodium hydroxide (10% by weight in water) | | 0.4% |
| D | Tocopheryl Acetate | | 1.0% |
| | Fragrance | | 0.2% |
| | Preservative | | q.s. |
| E | Dihydroxyacetone | | 5.0% |
| | Water | | 8.0% |

### Production:

I Melt component A at about 80° C.
II Stir B into A.
III Add C and cool down with stirring.
IV Stir D into III at about 30° C.
V Dissolve dihydroxyacetone in water and add E to IV.
VI If necessary, adjust pH to about 4.
Viscosity (Brookfield, 20° C., 20 revolutions/minute): 29 000 mPa·s.

### Formulation Example 2

### Self-Tanning Gel

| | | |
|---|---|---|
| A | Dihydroxyacetone | 5.0% |
| | 1,2-Propanediol | 2.5% |
| | Sorbitol F liquid | 2.5% |
| | Methyl 4-hydroxybenzoate | 0.2% |
| | Demineralized water | 28.3% |
| B | Sodium Acryloyldimethyltaurate/VP Copolymer | 1.5% |
| | (crosslinked) | |
| | Demineralized water | ad 100% |

### Production:

I Add sodium acryloyldimethyltaurate/VP copolymer to water with vigorous stirring.
II Dissolve dihydroxyacetone in water and add components A in succession with stirring.
III Add I and II together and homogenize.

1. A method of producing a polymer containing
a) at least one structural repeat unit of formula (1) 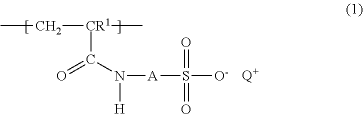
wherein R¹ is hydrogen, methyl or ethyl, A is C₁-C₈-alkylene, Q⁺ is H⁺, NH₄⁺, Na⁺, K⁺, ½Mg⁺⁺, ½Ca⁺⁺ or ½Zn⁺⁺ subject to the proviso that in from 50 to 100 mol %, of the structural units of formula (1) Q⁺ is not H⁺ or NH₄⁺,
b) optionally at least one structural repeat unit of formula (2) 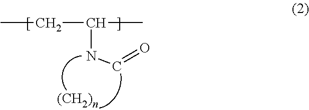
wherein n is an integer from 2 to 9,
c) optionally at least one structural repeat unit of formula (3) 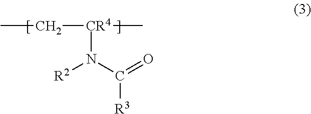
wherein R², R³ and R⁴ may be the same or different and are each hydrogen, a linear or branched alkyl group having from 1 to 30, carbon atoms or a linear or branched mono- or polyunsaturated alkenyl group having from 2 to 30 carbon atoms,
d) optionally at least one structural repeat unit derived from monomers of formula (4) 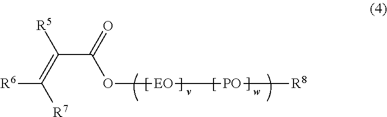
wherein R⁵ is H or —CH₃, R⁶ and R⁷ are each independently H or —CH₃, R⁸ is a linear or branched alkyl group having from 1 to 30 carbon atoms, a linear or branched mono- or polyunsaturated alkenyl group having from 2 to 30 carbon atoms, or a cycloaliphatic, arylaliphatic or aromatic hydrocarbyl radical having from 6 to 30 carbon atoms, EO is an ethylene oxide unit, PO is a propylene oxide unit, v and w are each independently from 0 to 500, the sum total of v and w is on average ≧1, and the distribution of the EO and PO units across the -(-[EO]ᵥ-[PO]_{w}-)- group can be random, blocklike, alternating or gradientlike, and
e) optionally at least one crosslinking structural unit derived from monomers having two or more olefinic double bonds,
with the proviso that the polymer contains at least one structural unit selected from the group consisting of b), c), d), and e),
wherein the method comprises the steps of suspending the corresponding salts of these polymers, wherein the Q⁺ cations in the structural units of formula (1) are NH₄⁺ and additionally, where appropriate, H⁺, as a starting material in an organic solvent and adding at least one base containing Na⁺, K⁺, Mg⁺⁺, Ca⁺⁺ or Zn⁺⁺ ions to effect an ion exchange.

2. A method as claimed in claim 1, wherein the starting material contains from 50 to 100 mol % of the structural units of formula (1) with NH₄⁺ as counterion and from 0 to 50 mol % of the structural units of formula (1) with H⁺ as counterion.

3. A method as claimed in claim 1, wherein the starting material contains 100 mol % of the structural units of formula (1) with NH₄⁺ as counterion.

4. A method as claimed in claim 1, wherein the organic solvent is a protic solvent.

5. A method as claimed in claim 1, wherein the organic solvent is tert-butanol.

6. A method as claimed in claim 1, wherein the bases are selected from the group consisting of hydroxides and alkoxylates.

7. A method as claimed in claim 1, wherein the bases are selected from the group consisting of hydroxides and alkoxylates of Na⁺, K⁺ and/or Zn⁺⁺.

8. A method as claimed in claim 1, wherein the base is NaOH.

9. A method as claimed in claim 1, carried out at a temperature in the range from 25 to 100° C.

10. A method as claimed in claim 1, further comprising the step of passing an inert gas through the organic solvent.

11. A method as claimed in claim 1, carried out at a pH in the range from 7.5 to 8.5.

12. A method as claimed in claim 1, wherein the at least one structural unit of formula (1) is derived from 2-acrylamido-2-methylpropanesulfonic acid.

13. A method as claimed in claim 1, wherein the at least one structural unit of formula (2) is derived from N-vinylpyrrolidone.

14. A method as claimed in claim 1, wherein R², R³ and R⁴ in the at least one structural unit of formula (3) are each independently hydrogen or methyl.

15. A method as claimed in claim 1, wherein R⁸ in the at least one structural unit derived from the monomers of formula (4) is a linear or branched alkyl group having from 8 to 22 carbon atoms, or is a linear or branched mono- or polyunsaturated alkenyl group having from 8 to 22 carbon atoms.

16. A method as claimed in claim 1, wherein the sum total of v and w in the structural unit derived from the monomers of formula (4) is on average in the range from 1 to 40.

17. A method as claimed in claim 1, wherein the polymers do not contain any crosslinking structural units of component e).

18. A method as claimed in claim 1, wherein the polymers contain up to 8% by weight of one or more crosslinking structural units of component e).

19. A method as claimed in claim 1, wherein the polymer contains
a) from 69.5% to 97.5% by weight of at least one structural repeat unit of formula (1),
b) from 2% to 30% by weight of at least one structural repeat unit of formula (2), and
e) from 0.2% to 3% by weight of at least one crosslinking structural unit derived from monomers having two or more olefinic double bonds.

20. A method as claimed in claim 1, wherein the polymer contains
a) from 49.99% to 98.99% by weight of at least one structural repeat unit of formula (1),
d) from 1% to 50% by weight of at least one structural repeat unit derived from the monomers of formula (4), and
e) from 0% to 8% by weight of at least one crosslinking structural unit derived from monomers having two or more olefinic double bonds.

21. A polymer obtainable by a method as claimed in claim 1.

22. A The thickener or stabilizer, comprising a polymer as claimed in claim 21.

23. A thickener or stabilizer as claimed in claim 22 wherein the pH is adjusted to a value above 7.

24. An alkaline lotion or cream for smoothing the skin or in alkaline hair colorants, comprising a-thickener or stabilizer as claimed in claim 22.

25. (canceled)

26. A self-tanning agent containing at least one polymeras claimed in claim 21 and from 0.1% to 10% by weight of dihydroxyacetone, based on the final agent, wherein this self-tanning agent is adjusted to a pH in the range from 3.5 to 5.5.
